(19) United States
(12) Patent Application Publication
(10) Pub. No.: US 2009/0036417 A1
(43) Pub. Date: Feb. 5, 2009

(54) COCHLEATES WITHOUT METAL CATIONS AS BRIDGING AGENTS

(76) Inventor: Tuo JIN, Highland Park, NJ (US)

Correspondence Address: LAW OFFICES OF ALBERT WAI-KIT CHAN, PLLC, WORLD PLAZA, SUITE 604, 141-07 20TH AVENUE, WHITESTONE, NY, 11357, US

(21) Appl. No.: 12/239,847

(22) Filed: Sep. 29, 2008

Related U.S. Application Data
(63) Continuation of application No. 10/636,522, filed on Aug. 6, 2003, now abandoned.
(60) Provisional application No. 60/425,825, filed on Nov. 13, 2002.
(60) Provisional application No. 60/401,686, filed on Aug. 6, 2002.

(51) Int. Cl. A61K 31/555; A61K 31/28; C07F 5/00; A61P 43/00

(52) U.S. Cl. 514/185; 514/492; 534/15

(57) ABSTRACT

This invention provides a cochleate and nano-cochleate systems wherein the agents bridging lipid bilayer are organic multi-valent cations. This invention also provides a method for preparing the cochleate system comprising direct cochleation and hydrogel-isolated procedure. The preparation method comprises using the charge ration between the bridging agents and lipids to control the particle sizes. This cochleate or nano-cochleate system may be used for microencapsulation and delivery of therapeutics wherein the therapeutic agents are loaded in the cochleate structure as the bridging agents between lipid bilayers. Finally, this invention provides other uses of these new cochleate and nano- cochleate systems.

### CROSS-REFERENCE TO RELATED APPLICATION

This application claims priority of U.S. Ser. No. 60/401,686, filed on Aug. 6, 2002, and U.S. Ser. No. 60/425,825, filed on Nov. 13, 2002, the contents of which are incorporated here into this application.

Throughout this application, various references are referred to and disclosures of these publications in their entireties are hereby incorporated by reference into this application to more fully describe the state of the art to which this invention pertains.

### FIELD OF THE INVENTION

The present invention demonstrates a novel phospholipid composition and its application in delivering various therapeutic agents to tissue and/or membranes which are impermeable. This composition comprises negatively charged lipid bilayers which interact with organic-multi-cations to roll up, forming a cylindrical multi-layer structure.

### BACKGROUND OF THE INVENTION

While the progress of biotechnology has brought more and more biological therapeutics to clinical applications, development of appropriate dosage forms for these agents are far behind the pace of development of the agents. Owing to their tissue impermeability and in vivo instability, most biological therapeutic agents are administered by frequent injection [1], that results in poor patient compliance.

Advanced drug delivery systems for biological agents have attracted considerable research efforts in recent years [2].

Among various drug delivery routes, oral delivery is, by far, the easiest and most convenient method for drug administration, especially when repeated doses and long therapeutic periods are necessary. Many approaches have been reported for oral delivery of tissue-impermeable drugs [3]. Strategies for improving oral absorption may be divided to i) converting a drug to lipophilic pro-drug, ii) conjugating a drug with lipophilic moieties, and iii) encapsulating a drug into particulate systems [3]. Particulate systems may offer good protection of delicate biological agents with no need for chemical modification of the molecules selves. However, absorption of particles by the intestines is generally less than 1% [3].

Because of the structural similarity of liposomes (phospholipid bilayer vesicles) to cellular membranes, the material had once been regarded as an ideal system for delivering therapeutics and attracted considerable research efforts since its discovery roughly four decades ago [4]. However, their physicochemical and biological instability retarded their success in practical application of liposomes in drug therapy. There are only limited liposome-based formulations which are commercially available despite R & D efforts [5]. To overcome this stability problem, several alternative lipid bilayer systems have been reported, namely, stealth™ liposomes [5], polymerized liposomes [6], polyethylene glycol coated liposomes [7], lipo-beads [8], and cochleates [9].

Cochleates are spiral rolls formed of negatively charged phospholipid bilayers which are rolled up through the interaction with multivalent counter ions (Ca²⁺ or Zn²⁺) as bridging agents between the bilayers [9]. As a particulate system, cochleates possess unique properties in that they offer superior mechanical stability and better protection for encapsulated drugs compared with liposomes due to their solid matrix. Cochleates also maintain their phospholipid bilayer structures. These solid particles are so flexible that they can readily convert to liposomes by extracting the bridging counter ions out of the inter bilayer spaces. Such unique properties have made cochleates an ideal system for delivering insoluble ingredients which can be loaded in the matrix of a phospholipid bilayer while avoiding the instability problem of liposomes [10].

Recently, another patented technology that demonstrated structure, preparation and application of nanometer-sized cochleates (nanocochleates) [11]. These size-reduced cochleates show a capability for oral delivery of Amphoterecin B (AmB), a hydrophobic drug currently administrated through IV injection in the form of liposomes or micelles. Oral availability of AmB achieved by nanocochleates encouraged the design of a new cochleate system by which highly charged and membrane-impermeable therapeutics may be encapsulated and delivered orally.

In the case that cochleates are used as drug carriers, hydrophobic drug molecules are incorporated in the matrix of the phospholipid bilayer prior to cochleation (formation of cochleates by addition of metal cations). Drug loading capacity is limited by how much drug can be “dissolved” in the lipid matrix without destroying its bilayer structure. This structure limits application of cochleates to deliver of hydrophobic molecules.

It has been reported that cochleates were used to deliver DNA and protein vaccines [12, 13]. It has been suggested that these hydrophilic macromolecules were incorporated in the cochleate structure. However, there was no solid evidence presented. The only experimental observation was that the concentration of DNA or proteins in the supernatant was reduced after the addition of calcium ions. Since the multivalent ions (Ca²⁺ or Zn²⁺) used in cochleate formation can also complex with proteins and DNA (Both are charged polymers.) through ionic interaction, the likelihood is that these macromolecules interacted with Ca²⁺ or Zn²⁺ and were merely precipitated at the same time when cochleates were forming. Owing to the size and hydrophilic nature of these macromolecules, encapsulation of proteins and DNA into the lipid bilayer matrix or the inter-bilayer space is unlikely.

In the present invention, a new approach that ensures that hydrophilic drugs are loaded in the inter-bilayer space of cochleates and nanocochleates is demonstrated.

### SUMMARY OF THE INVENTION

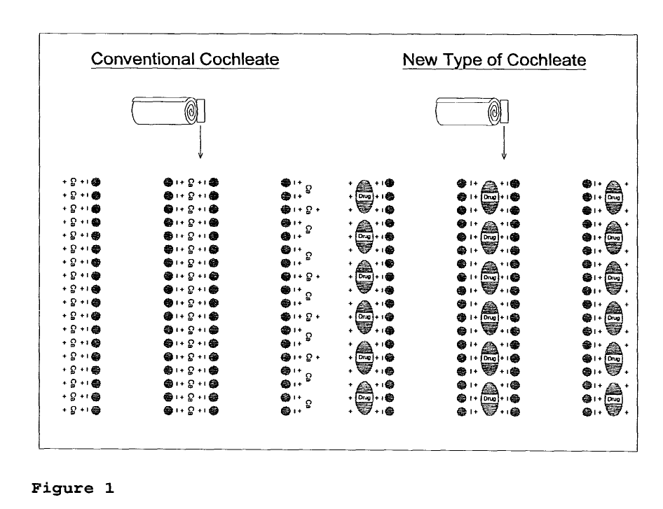

The present invention demonstrates a new type of cochleate and nano-cochleate that allow charged, soluble but tissue-impermeable molecules, including relatively small therapeutic peptides, to be encapsulated in the inter-bilayer space and delivered a cross tissue-membrane. Cochleates and nano-cochleates are phospholipid-calcium (or zinc) precipitates that are formed by calcium- (or zinc-) induced fusion of unilamellar liposomes into large lipid bilayer sheets which then fold spirally into cylinders. The new cochleates and nano-cochleates differ from conventional systems in that i) the fusion of unilamellar liposomes is no longer induced by Ca²⁺, Zn²⁺ or other metal ions but by the molecules to be encapsulated (See FIG. 1.); ii) charged, hydrophilic and tissue-impermeable drugs can be encapsulated in the structure with improved loading capacity. Since no additional metal cations (such as Ca²⁺ or Zn²⁺) exist during the new cochelation process, there is no possibility that the molecules to be encapsulated are precipitated outside of the cochleate structure as in conventional cochelation.

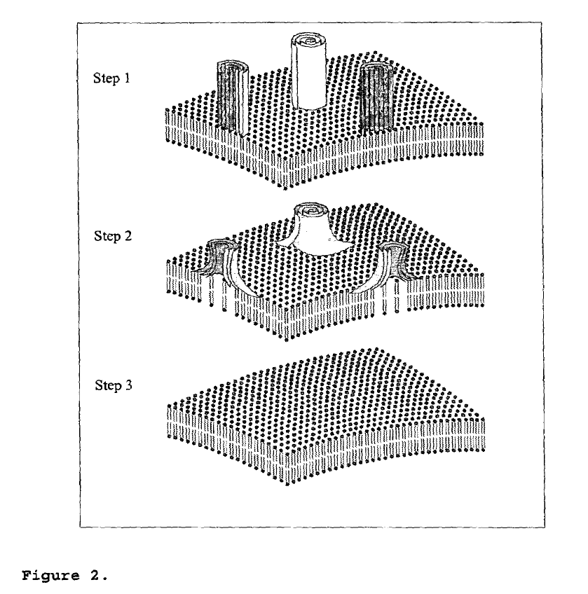

On the other hand, the new cochleates and nano-cochleates showed some similarities in physical chemical properties and drug delivery functions as the conventional systems. The cylindrical structure could open up and convert to liposomes upon the addition of a cation carrier, such as EDTA. In addition, the new system showed the ability to deliver encapsulated ingredients across cell membranes by fusion with the membrane (See FIG. 2.).

Organic cations of various sizes (2,3,5,6-tetraaminopyrimidine, tobramycin, and polylysine) were examined in the present invention, and the cylindrical cochleate structure was observed in all cases. These results suggest a wide flexibility in formation of cochleates with organic cations as the bridging agents between the lipid bilayers.

This invention offers a simplified method for preparing nano-cochleates. When poly-cations (such as polypeptides with a net charge over 5) were used, nano-cochleates were easily prepared by adding the polycations directly into the lipsomal suspension, without using complicated hydrogel-isolation technique.

### DETAILED DESCRIPTION OF THE FIGURES

FIG. 1. Schematic description of complexation of phospholipids bilayers with Ca²⁺ and with organic cations.

FIG. 2. Schematic description of fusion of cochleates formed by interaction with drug molecules which function as the bridging agent between phospholipid bilayers. The loaded drug molecules are delivered across cell membranes due to fusion of cochleates with the cell membrane.

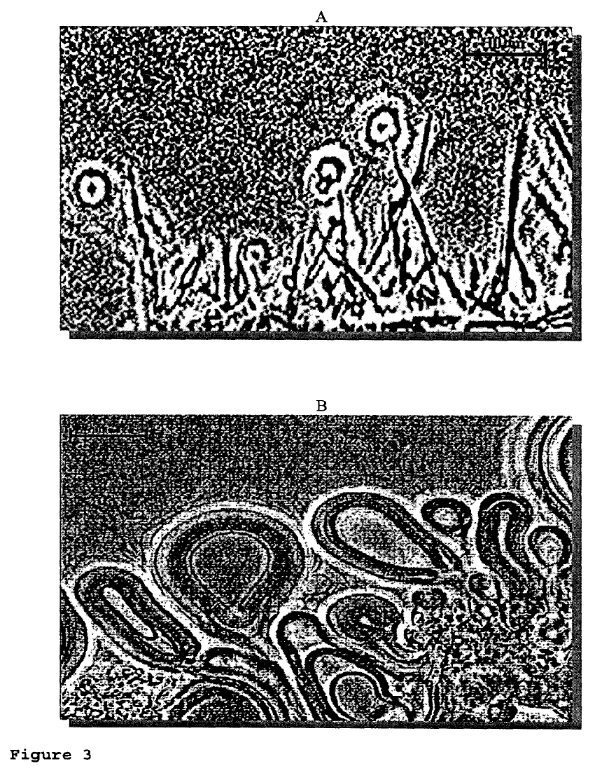

FIG. 3. Microscopic image of cochelates formed by complexation with 2,3,5,6-tetraaminopyrimidine as the bridging agent. A: before treatment with EDTA; B: after treatment with EDTA.

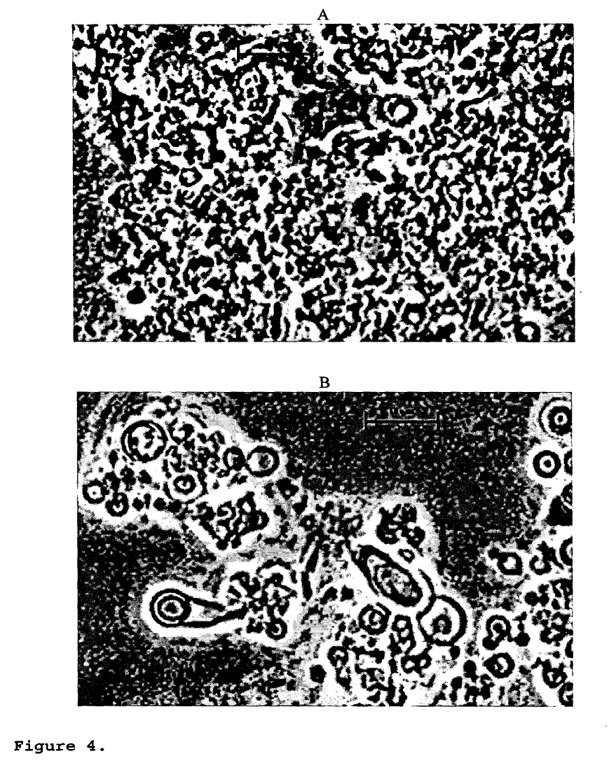

FIG. 4. Microscopic image of nano-cochelates formed by complexation with 2,3,5,6-tetraaminopyrimidine as the bridging agent. A: before treatment with EDTA; B: after treatment with EDTA.

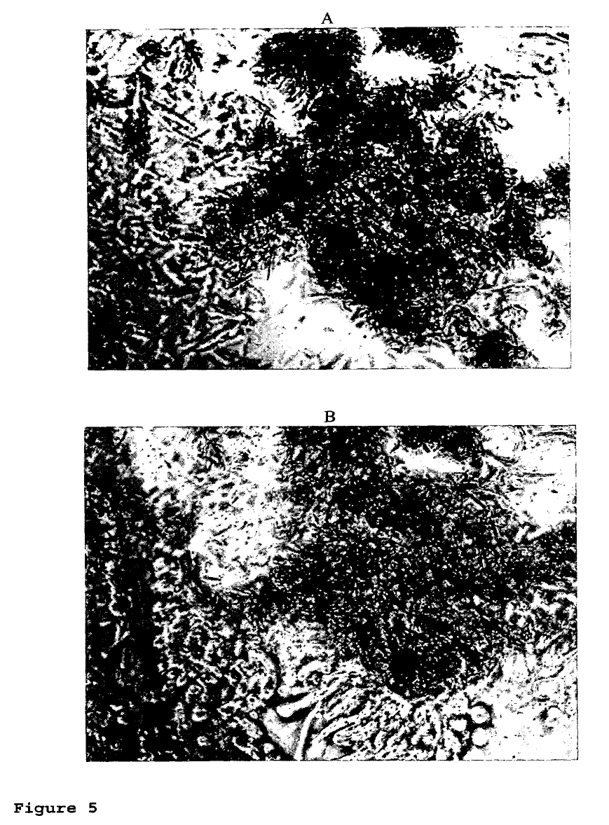

FIG. 5. Microscopic image of cochelates formed by complexation with tobramycin as the bridging agent. A: before treatment with EDTA; B: after treatment with EDTA.

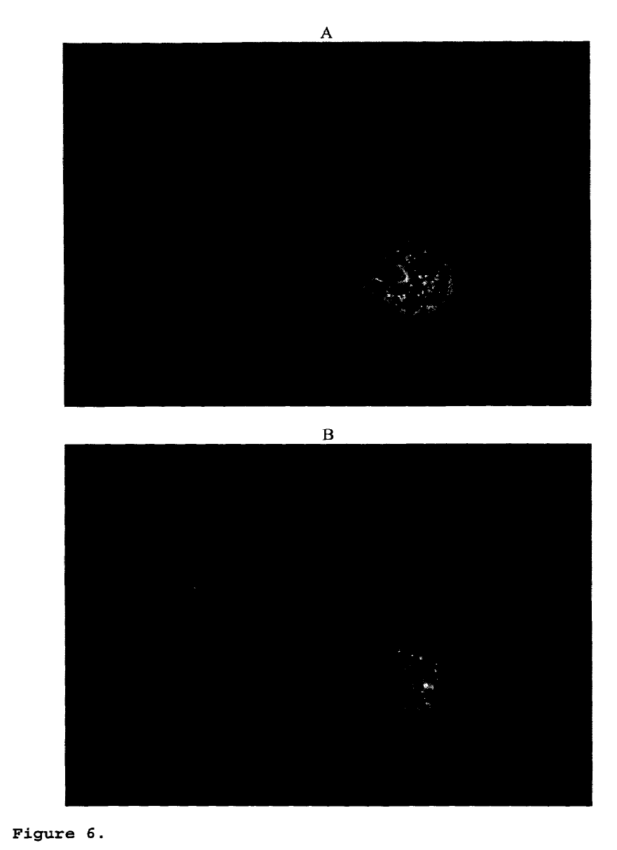

FIG. 6. Microscopic image of nano-cochelates formed by complexation with tobramycin as the bridging agent. A; before treatment with EDTA; B: after treatment with EDTA.

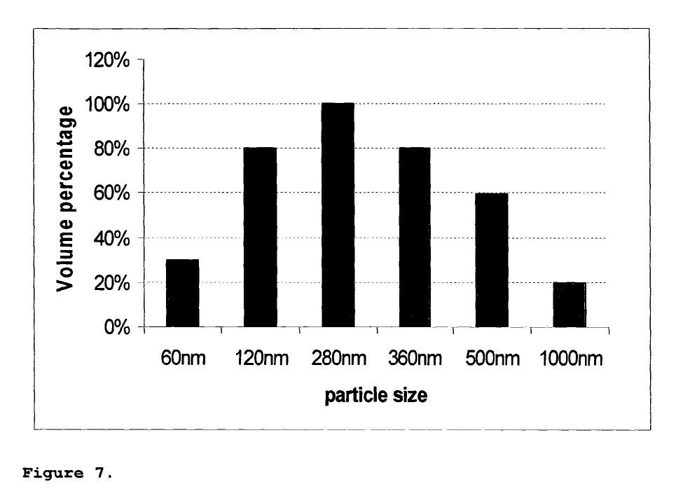

FIG. 7. Distribution of dynamic sizes of nano-cochleates formed by complexation with tobramycin.

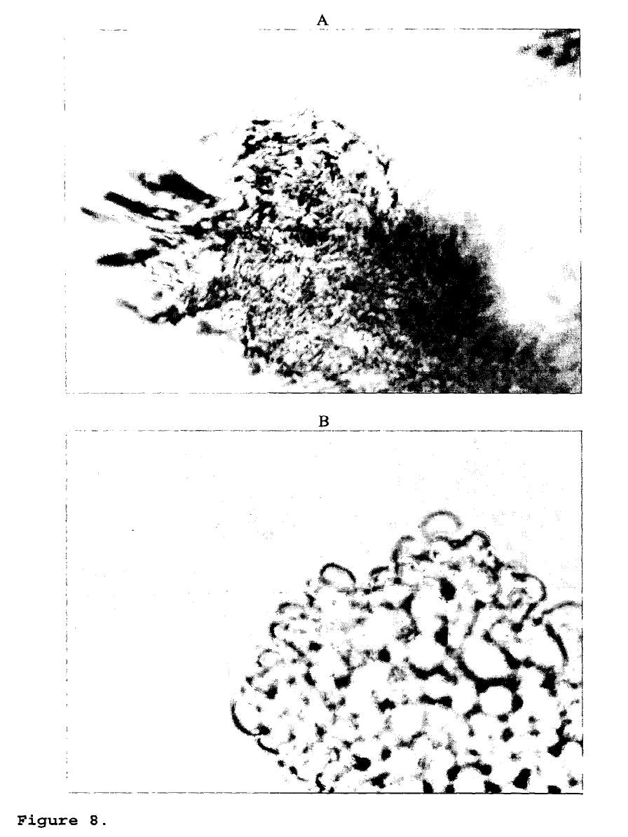

FIG. 8. Microscopic image of cochelates formed by complexation with polylysine as the bridging agent. A: before treatment with EDTA; B: after treatment with EDTA.

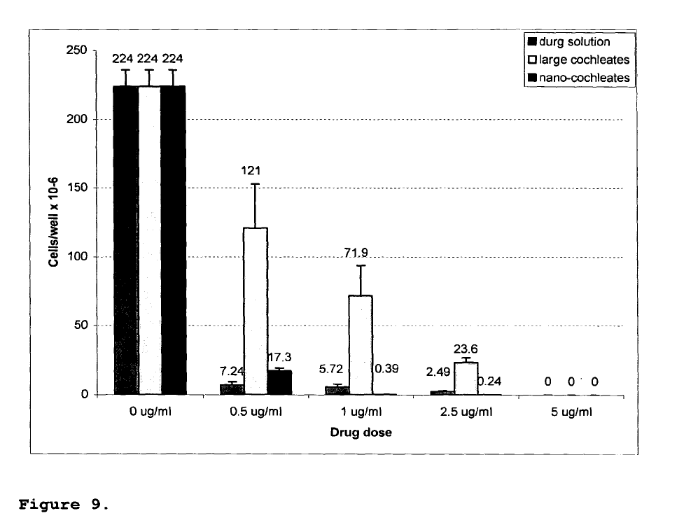

FIG. 9. Antibiotic activity of tobramycin formulated in solution, cochleates and nano-cochleates. The drug of various doses were added to E. Coli prior to incubation at 37° C., followed by counting of the colonies.

### DETAILED DESCRIPTION OF THE INVENTION

This invention provides a new cochleate system and a nano-cochleate system for which the agents that bridge lipid bilayers together to form a multi-layer structure are organic multi-valent cations. As used herein, the new cochleate systems are defined as a spiral phospholipids bilayer that rolled up by complexation with organic multi-valent cations which bring two surfaces of charged lipid bilayers together through ionic bonds (See FIG. 1.). The multi-bilayer systems formed by interaction with the organic cations may or may not form a cylindrical shape.

The new systems can allow charged and hydrophilic therapeutics, such as peptides, to be microencapsulated into the cochleate structure while conventional cochleates cannot. On the other hand, however, the new systems showed properties similar to those observed in conventional cochleates, such as conversion back to liposomes when treated with cation carriers and the ability to deliver drugs across tissue membranes. These properties (loading hydrophilic drugs and delivery across membrane) make the new systems promising for oral delivery of peptides.

The cochleate and nano-cochleate systems disclosed herein can be used for microencapsulation and delivery of therapeutics, wherein the therapeutic agents are loaded in the cochleate structure as the bridging agents between lipid bilayers.

The therapeutics include, but are not limited to, peptides, poly-amino acids, nucleotides, and hydrophilic chemical drugs which possess two or more net charges. Other drugs may be used. An ordinary skilled artisan may use the drugs exemplified herein or the guidelines provided in other drugs.

In an embodiment, the above-described cochleate systems are used for oral delivery of peptides, polyamino acids, nucleotides, and hydrophilic chemical drugs which possess more than two net positive charges.

In another embodiment, the delivery of therapeutics is through inhalation.

This invention also provides a method of preparing the new cochleate systems comprising direct cochleation [9], hydrogel-isolated cochleation [11], and size-controlled cochleation using poly-cations. See e.g. Example 5.

Organic cations can be added to a suspension of unilamellar liposomes directly with stirring or vortex, or added to polymer aqueous two-phase system for which liposomes are partitioned in the dispersed phases and isolated within each droplet [11].

Another advantage of this invention is that nano-cochleates can be prepared without using the complicated hydrogel-isolation technique [11]. For those polycations which possess multiple net charges, the sizes of cochleates formed can be controlled by the charge ratio of the polycations over liposomes. In a preferred embodiment, the charge is more than five. The size of cochleate is about 40 nm to about 1000 nm in dynamic diameter. By increasing the polycations over stoichiometrical amount, nano-cochleates can be formed by adding the polycations directly to the liposomal suspension. See e.g. Example 5. Owing to their multiple net charges and long chain, polycations may be partially associated with the lipids of opposite charge, leaving some charged species dangling at the cochleate surfaces. This invention offers a significantly simplified method to prepare nano-cochleates.

This invention provides a composition comprising a cochleate system, wherein the agents bridging lipid bilayer are organic multivalent cations. This invention further provides a pharmaceutical composition comprising the above-described cochleate system and a pharmaceutically acceptable carrier.

For the purposes of this invention, “pharmaceutically acceptable carrier” means any of the standard pharmaceutical carriers. Examples of suitable carriers are well known in the art and may include, but are not limited to, any of the standard pharmaceutical carriers such as a phosphate buffered saline solution and various wetting agents. Other carriers may include additives used in tablets, granules and capsules, etc. Typically such carriers contain excipients such as starch, milk, sugar, certain types of clay, gelatin, stearic acid or salts thereof, magnesium or calcium stearate, talc, vegetable fats or oils, gum, glycols or other known excipients. Such carriers may also include flavor and color additives or other ingredients. Compositions comprising such carriers are formulated by well-known conventional methods.

This invention provides a method to treat a subject with a disease comprising administering to the subject the above-described cochleate system which comprises an appropriate drug for said disease. In an embodiment, the subject is a mammal. In a further embodiment, the subject is a human.

The invention will be better understood by reference to the examples which follow, but those skilled in the art will readily appreciate that the specific experiments detailed are only illustrative and are not meant to limit the invention as described herein, which is defined by the claims which follow thereafter.

### EXAMPLES

### Example 1

Preparation of Cochleates with 2,3,5,6-tetraaminopyrimidine sulfate

A multivalent organic cation, 2,3,5,6-tetraaminopyrimidine sulfate (TAS), was dissolved in water with a concentration of 10.5 mM and pH of 2 (adjusted with HCl). A suspension of small unilamellar liposomes (SUV) was prepared by suspending dioleoyl phosphatidyl serine (DOPS) in water, followed by sonication in a N₂ atmosphere. The lipid-water suspension looked milky at the beginning, but turned clear (with a slightly blue tint) as sonication proceeded. The sample was examined using an optical microscope, and no liposome was observed, indicating that liposomes are smaller than 1 micron.

To prepare cochleates, the TAS solution was added to the liposome suspension drop-wise under magnetic stirring until precipitation occurred. The precipitates were examined using a microscope, and the microscopic image showed that the lipids formed needle-shape structures (See FIG. 3A.). Other organic cationic molecules, such as antibiotics and polypeptides, can also be used to form cochleate structure.

To examine properties of the new cochleates, a drop of EDTA (200 mM, pH 8.5) was added to the precipitates loaded on a microscope slide and observed under the microscope. As shown in FIG. 3B the needle shape precipitates opened up and converted to giant liposomes. This is a typical property of conventional cochleates. The results in FIG. 3A and 3B indicate that organic multivalent cations interact with liposomes (made of DOPS) to form cochleates as those formed with Ca²⁺ and Zn²⁺.

### Example 2

Preparation of Nano-Cochleates with 2,3,5,6-tetraaminopyrimidine sulfate.

Nano-sized cochleates can be prepared with 2,3,5,6-tetraaminopyrimidine sulfate using previously patented hydrogel-isolated methods [11]. In brief, a liposome suspension prepared as in Example 1 was added into a dextran solution (5-25%) with a lipid content of 0.2-2%. This suspension was then dispersed into a polyethylene glycol (PEG) solution (5-25%) and well stirred. The solutions of dextran and PEG were immiscible and formed an aqueous two-phase system. The TAS solution prepared as in Example 1 was added drop-wise to the aqueous two-phase system under stirring and the charge of the organic cations was more than that of the lipids. The aqueous two-phase system was stirred for an additional 10 to 60 min, and then the cochleates formed were recovered by rinsing the dextran and PEG away using a sufficient amount of water (by which dextran and PEG were all dissolved in one phase), followed by centrifugation. FIG. 4A show a microscopic image of recovered cochleates. Needle structure was not detectable by optical microscope due to particle size. A laser scattering measurement showed that the cochleate sizes were sub-micron. FIG. 4B shows the microscopic image of nano-cochleates after treatment with EDTA. Liposomes formed from nano-cochleates are much smaller than those from cochleates formed without hydrogel-isolation (Compare FIG. 3B with FIG. 4B.). A similar result indicating that EDTA treatment of nano-cochleates only generated small liposomes was reported in a previous invention of calcium- induced nano-cochleates [11]. The particle size distribution of nano-cochleates was measured using a Nicomp submicron particle sizer, showing the mean dynamic size was around 400 nm.

### Example 3

Preparation of Cochleates and Nano-Cochleates with Tobramycin

Cohcleates and nano-cochleates were prepared by repeating the experimental procedure in Examples 1 and 2 using a drug, tobramycin chloride, as the bridging agent instead of TAS. Tobramycin is an antibiotic, soluble in water in salt form and administrated by injection. The molecule has a molecular weight of 467 and 5 amino groups.

To prepare cochleates, a solution of tobramycin was prepared by dissolving 100 mg tobramycin with 100 ml water. Prior to cochleation, the solution was divided into several parts with pH adjusted to 1.2, 2.5, 3.5, and 5, respectively. These drug solutions were added dropwise to liposome solutions prepared as in Example 1, respectively. Visible precipitates were formed for the samples treated with tobramycin solution with pH of 1.2 and 2.5, suggesting that sufficient ionization of the amino groups of tobramycin is required. The formed cochleates and their response to EDTA were examined using an optical microscope. The images were shown in FIGS. 5A and 5B, respectively. The precipitates showed needle shapes (FIG. 5A) before treatment with EDTA, and converted to giant liposomes when EDTA was added (FIG. 5B).

To prepare nano-cochleates with tobramycin, the procedures of Example 2 were followed, with the TAS solution replaced by the tobramycin solution with pH=2.5. The images of nano-cochleates under an optical microscope are shown in FIG. 6A. Similarly, addition of EDTA caused conversion of nano-cochleates to small liposomes (FIG. 6B). The particle size distribution of the nano-cochleates was measured using a Nicomp submicron particle sizer, showing the mean dynamic size was around 300 nm (FIG. 7).

### Example 4

Preparation of Cochleates with Polylysine

Cochelates can also be prepared by adding a peptide solution into the liposomal suspension as in Example 1. When a solution of polylysine (MW=1000˜2000, pH=4) was added drop-wise to the same liposomal suspension as in Example 1, precipitates were formed. The final ratio of DOPS and polylysine was 1:1.2. A microscopic image showed that the precipitated particles possess a needle shape (FIG. 8A). These needle-shaped particles readily opened up and converted to giant liposomes (FIG. 8B) as those prepared with other bridging agents (Ca²⁺[11], Zn²⁺[11], TAS, and tobramycin).

### Example 5

Preparation of Nano-Cochleates with Polylysine

Nanometer sized cochleates can be prepared with peptides as the bridging agent without using the hydrogel-isolation [11]. In this experiment, the liposomal suspension prepared as in Example 1 was added into the polylysine solution as in Example 4 under stirring with the final lipid to polylysine ratio of 1:4. The clear liquids (polylysine solution and liposomal suspension) readily turned cloudy. No visible particles were observed under optical microscope. A particle size measurement was carried out using a Nicomp Submicron particle sizer, suggesting the mean dynamic size of the particles was about 60-100 nm.

The mechanism of the size reduction due to the increased polylysine-to-liposome ratio may be similar to that in the complex formation between DNA and cationic polymers [14].

### Example 6

Loading Capacity of Cochelates for Molecules as Bridging Agents.

Loading capacities of the new cochelates and nano-cochleates for TAS and tobramycin were determined.

For TAS, known amounts of cochleates or nano-cochleates were first dissolved with chloroform, then added with water (pH=2), followed by strong shaking. The water phase was separated hereafter, and the same procedure was repeated two more times. The water phases collected three times were combined. The concentration of the extract was determined by UV absorption at 380 nm.

For tobramycin, since the molecule is UV-inactive, a HPLC method based on a reaction with a dye (a USP method) was used. The loading amount was determined based on the decreased concentration in the supernatant after cochleate formation.

For mobile phase, 2.0 g of tris (hydroxymethyl) aminomethan was first dissolved with 800 ml of water, followed by addition of 20 ml of 1N H₂SO₄ and acetanitrile to a total volume of 2000 ml.

For detectability, a solution of 2,4-dinitrofluourobene (10 mg/ml in alcohol) was prepared within 5 days prior to use and refrigerated. A water solution of tris (hydroxymethyl) aminomethane (15 mg/ml) was also prepared as a stock solution. Within 4 hours of analysis, this stock solution, 40 ml, was diluted with dimethyl sulfoxide (DMSO) to 200 ml.

For the standard curve, 550 mg of tobramycin and 2 ml of 1N H₂SO₄ were dissolved in water to make a solution of 100 ml in volume. Prior to analysis, this solution was further diluted 5 times with the tobrmycin content 0.22 mg/ml of tobramycin.

For the supernatant (the sample to be measured), 1 ml of 5.5 mg/ml of tobramycin solution was added into a liposome suspension with a lipid-to-drug ratio of 1:5. After precipitation, the supernatant was collected and diluted to 50 ml with water.

5 ml of the pre-prepared 2,4-dinitrofluourobene solution and 5 ml of the pre-prepared tris (hydroxymethyl) aminomethane solution were added to the standard and sample (the supernatant) solutions of tobramycin, each 2 ml. Then the standard and the sample were allowed to react with the added agents at 60° C. for 50 min. After cooling down, the two samples were diluted by acetonitrile to 25 ml.

An acetonitrile solution of P-naphtholbenzein, at a ratio of 4:1 was added to the treated standard and the sample prior to HPLA measurement. HPLC analysis was carried out using a C-18 column with absorption selected at 266 nm.

The loading capacity for TAS and tobramycin are listed in Table 1.

TABLE 1
Loading capacity of cochleates for 2,3,5,6-
tetraaminopyrimidine sulfate and tobramycin.

| | Lipids | Drug | Drug/Lipid |
|---|---|---|---|
| Model Drug | (mg) | (mg) | (mole/mole) |
| TAPS* | 5 | 0.0248 | 1.0/2.0 |
| Tobramycine | 38.11 | 5.5 | 1.0/4.0 |

### Example 7

Antibiotic Activity of Tobramycin Loaded in Cochleates and Nano-Cochleates.

Tobramycin was selected as a model drug to examine the capability of cochelates and nano-cochleates to deliver hydrophilic drugs across cell membranes because the antibiotic function of tobramycin relies on its binding to ribosomes inside of cells. In other words, tobramycin's antibiotic activity reflects internalization of the drug into the cells. For this purpose, tobramycin-loaded cochleates and nano-cochleates prepared as in Example 2 were incubated with E. Coli at various doses. As a control, a tobramycin solution was also incubated with E. Coli under identical conditions.

Experimentally, one drop of E. Coli cell line, DH5α, was added to 2 ml LB Broth solution and incubated at 37° C. for 24 hrs to prepare 1:10 DH5α culture solution. Then, 5 ul of the DH5α culture solution was diluted to 2 ml and tobramycin was added in the form of cochleates and nano-cochleates at the final concentrations of 0, 0.5, 1.0, 2.5, and 5.0 μg/ml, respectively. The tobramycin-added cell cultures were incubated at 37° C., with shaking at 200 rpm, for 24 hrs. The incubated cell culture suspension was then diluted by 1:100, 1:1000, and 1:10000 times, and plated as 50 ul diluted cultures to each agar dish. The dishes were further incubated at 37° C. for another 24 hrs prior to the counting of the colonies. The result is shown in FIG. 9.

At low doses (0.5 μg per 1 ml of medium, i.e. 0.5 μg/ml), the drug solution showed the highest activity, 75-times higher than that of large cochleates and slightly higher than nano-cochleates. With the dose increased to above 1 μg/ml (the dose suggested by USP), nano-cochleates became the most active dosage form. The cell counts for nano- cochleate treated culture was ten times lower than that by tobramycin solution and 100 times lower than that of large cochleates. At the dose of 5.0 μg/ml, cell counts became zero for all the three formulations. It is clear that nano- cochleates significantly enhanced antibiotic activity of the drug. Based on the loading capacity of the tobramycin and the average size of nano-cochleates, approximately 2,000,000 drug molecules are loaded in one nano-cochleate. Therefore, at the same tobramycin dose, the frequency for nano-cochleate particles colliding with the cells should be much lower than that of free drug molecules in the solution. Nevertheless, more drug molecules entered E. Coli cells in nano-cochleate form compared with the free solution form (FIG. 9). This result supports the hypothesis that the cochleate structure facilitated permeation of hydrophilic agents across cell membranes. The high tension at the edge of the phospholipid bilayer at the two ends of cochleate cylinders probably facilitated fusion of cochleates with cell membranes (FIG. 2).

At low dose (0.5 μg/ml), the drug solution showed slightly higher activity probably due to the fact that the number of nano-cochleate particles was too low for sufficient exposure of the cells (to the drug). The same reasoning also explains the relatively lower activity for large cochleates (FIG. 9).

The properties that nano-cochleates can facilitate cross-membrane diffusion for charged and impermeable molecules have a wide application in drug delivery. Many therapeutic agents, such as peptides, are soluble but impermeable to tissue membranes. Cross-membrane permeation is especially important for those agents for which the binding sites are inside of cells rather than cell surface receptors. The system may also facilitate oral absorption for peptide drugs that possess a net positive charge.

The nano-cochleates demonstrated in a previous invention [11] showed significant in vivo bioavailability and therapeutic efficacy for oral delivery of amphotericin B, a hydrophobic anti-fungus agent normally administrated through the IV route. The new nano-cochleates, although differing from the previous one by using organic cations as the bridging agents, possess similar physical chemical properties including the ability to fuse with cell membranes (Example 7). Therefore, the new system is expected to have oral bioavailability of impermeable therapeutics similar to that of amphotericin B delivered by the previously reported nanochleates [11].

### REFERENCES

1. P. Vermeij, D. Blok, “New peptide and protein drugs”, Pharm. World Sci., 1996; 18, 87˜93.

2. R. Langer, “Drug delivery and targeting”; Nature, 1998, 392 (Suppl.), 5-10.

3. H. Chen, R. Langer, “Oral particulate delivery: status and future trends”; Adv. Drug Delivery Rev., 1998, 14, 339-350.

4. G. Gregoriadis, in Liposome Technology, CRC Press, Boca Raton, Vol. I, 1993, Preface discussion.

5. G. Gregoriadis, in Liposome Technology, CRC Press, Boca Raton, Vol. I, 1993, Preface discussion.

6. H. Chen, V. Torchilin, and R. Carpenter, “Lectin-bearing Polymerized Liposomes as Potential Oral Vaccine Carriers,” Pharm. Res., 1996, 13(9), pp. 1378-1383.

7. T. Jin, P. Pennefather, P. I. Lee, “Lipobeads: a Hydrogel Anchored Lipid Vesicle System”; FEBS Letters, 1996, 397, 70-74.

8. D. Papahadjopoulos, “Calcium-induced phase changes and fusion in natural and model membranes,” Cell. Surg. Rev., 1978, 5, 765-790.

9. D. Papahadjopoulos, W. J. Vail, K. Jacobson, and G. Poste, “Cochleate lipid cylinders: formation by fusion of unilamellar lipid vesicles,” Biochim. Biophys. Acta, 1975, 394, pp. 483-491.

10. L. Zarif, J. Graybill, D. Perlin, R. J. Mannino; “Cochleates: new lipid based drug delivery system”; Journal of Liposoine Research, 2000, 10, 523-538.

11. T. Jin, L. Zarif, and R. J. Mannino, “Nanocochleate formulations, process of preparation and method of delivery of pharmaceutical agents”; U.S. Pat. No. 6,153,217, 2000.

12. S. Gould-Forgerite, J. E. Mazurkiewicz, K. Raska Jr., K. Voelkerding, J. M. Lehman, and R. J. Mannino, “Chimerasome-mediated gene transfer in vitro and in vivo,” Gene, 1989, 84, 429-438.

13. S. Gould-Forgerite, M. Kheri, F. Zhang, Z. Wang, A. J. Scolpino, E. Feketeova, M Canki, R. J. Mannino; “Targeting immune response induction with cochleate and liposome-based vaccines”; Adv. Drug Delivery Review, 1998, 32, 273-287.

14. A. V. Kabanov, V. A. Kabanov, “DNA complexes with polycations for the delivery of genetic materials into cells”, Bioconj. Chem., 1995, 6, 7-20.

1-16. (canceled)

17. A cochleate system comprising a water-soluble organic cation which is used to bridge negatively-charged lipid bilayers together to form a multi-layer structure, wherein the water-soluble organic cation possesses two or more net positive charges, and the water-soluble organic cation is an aminopyridine or an antibiotic.

18. The cochleate system of claim 17, wherein the multi-layer structure is cylindrical in shape.

19. The cochleate system of claim 17, wherein the cochleate is a nano-cochleate.

20. The cochleate system of claim 17, wherein the water-soluble organic cation is 2,3,5,6-tetraaminopyrimidine sulfate or tobramycin.

21. A method of preparing the cochleate system of claim 17, comprising adding the water-soluble organic cation to a liposome suspension directly or obtaining cochleation through a hydrogel-isolated procedure.

22. The method of claim 21, further comprising controlling cochleate size by adjusting a ratio of the water-soluble organic cation to lipids.

23. A composition comprising the cochleate system of claim 17.

24. The cochleate system of claim 17, wherein the cochleate system is used for delivering the water-soluble organic cation orally or through inhalation.

25. A cochleate system comprising a water-soluble organic cation which is used to bridge negatively-charged lipid bilayers together to form a multi-layer structure, wherein the water-soluble organic cation is 2,3,5,6-tetraaminopyrimidine sulfate or tobramycin.

26. The cochleate system of claim 25, wherein the multi-layer structure is cylindrical in shape.

27. The cochleate system of claim 25, wherein the cochleate is a nano-cochleate.

28. A method of preparing the cochleate system of claim 25, comprising adding the water-soluble organic cation to a liposome suspension directly or obtaining cochleation through a hydrogel-isolated procedure.

29. The method of claim 28, further comprising controlling cochleate size by adjusting a ratio of the water-soluble organic cation to lipids.

30. A composition comprising the cochleate system of claim 25.

31. The cochleate system of claim 25, wherein the cochleate system is used for delivering the water-soluble organic cation orally or through inhalation.
